(19) United States
(12) Patent Application Publication
(10) Pub. No.: US 2002/0193722 A1
(43) Pub. Date: Dec. 19, 2002

(54) Dressing product with alginate fibre core surrounded by a sheath

(76) Inventors: Philippe Maingault, Douai-La-Fontaine (FR); Michel Barikosky, Paris (FR)

Correspondence Address: Brown Raysman Millstein Felder & Steiner LLP, Attorney for Applicants, 900 Third Avenue, New York, NY, 10022, US

(21) Appl. No.: 10/142,568

(22) Filed: May 9, 2002

(30) Foreign Application Priority Data
May 11, 2001 (FR) 01 06252

(51) Int. Cl. A61F 13/00; A61F 15/00

(52) U.S. Cl. 602/43

(57) ABSTRACT

The dressing product comprises a metal alginate fibre core (1) surrounded by a supporting sheath (2) of a structure permeable to biological fluid and biocompatible with the wound. The core (1) and sheath (2) both extend into a continuous layer of alginate fibres and provide drainage slots (7, 8) between each other.

The product applies to moist and deep wounds.

The invention relates to a dressing product for biological fluid wounds, comprising a metal alginate fibre core surrounded by a supporting sheath of a structure permeable to the biological fluid and biocompatible with the wound.

Such a dressing product is particularly disclosed in EP 0 624 082.

The dressing product in question may be used for moist wounds, particularly deep wounds, such as exudative cavities, such as scars or other postoperative wounds. It may also be used in endonasal surgery, in the upper respiratory tract of the nasal fossa.

As the metal alginate, an alginate of a metal chosen in the family of multivalent metals is frequently considered, except for magnesium, and more particularly calcium alginate.

A wound induces a loss of substance, or of biological fluid (blood or exudate). Applied in a wound, the dressing starts by absorbing the exuding biological fluid, the water molecules of the fluid intercalating between the alginate macromolecules. Once it has swollen due to absorption, the dressing undergoes gelling by means of ion exchange. In the case of calcium alginate fibres, they give Ca²⁺ ions to the biological fluid which gives them Na⁺ ions. As the balance is established between the calcium and sodium, the alginate fibres partially lose their crystalline structure. The gelling of the dressing induces the drying of the wounds and prevents the adhesion of underlying tissue.

Despite the gelling, due to the supporting sheath, the dressing product still has a good mechanical cohesion and can be removed relatively easily from a wound in a painless manner.

However, the dressing product according to the prior art introduced above is not easy to manufacture.

The invention of the present application aims to propose one that is easy to manufacture.

To this end, the invention relates to a dressing product of the type defined above, characterised in that the core and sheath both extend into a continuous layer of alginate fibres and provide drainage slots between each other.

Formed from a single fibre layer, the dressing product is naturally easy to produce.

It is important to note that due to the unitary structure of the product according to the invention, the core also acts as a support or reinforcement of the sheath which prevents it from undergoing flattening or subsidence which would reduce the drainage slots. This is particularly advantageous in the treatment of cavities, before the impregnation of the fibres.

The fibre layer may be a non-woven, woven or knitted layer.

It should be pointed out that women's sanitary tampons, of the type described for example in U.S. Pat. No. 4,816,100, are also composed of a continuous core-sheath layer. However, the sheath does not provide any slots around the core since the purpose of the tampon is not to offer good exudate drainage capacities but, on the contrary, undergo expansion, which is also conical to block the cavity in a satisfactory manner. In fact, the core and sheath of these tampons do not at all perform the mutual support functions like those of the product according to the invention, provided firstly by the core for the sheath and then by the sheath for the core.

In the preferred embodiment of the product according to the invention, the sheath is composed of a tubular sheath wherein the edges adjacent to the selvedges are folded inwards to form the core.

Advantageously, the core extends at least over approximately a quarter of the diameter of the sheath, i.e. a quarter of the transversal dimension of the sheath.

Preferentially, the edges of the sheath of the dressing product according to the invention forming the core are oversewn, i.e. joined together by overhand sewing. In other words, the sheath is oversewn.

Preferentially, the overhand sewing extends longitudinally beyond the sheath and the core to form a pulling cord (string) to extract the dressing product after use, and for safety purposes, to prevent the dressing product from migrating during use. Preferentially again, the overhand sewing comprises two threads.

The invention will be understood more clearly using the following description of the dressing product and the equipment used to manufacture it, with reference to the appended figure, wherein:

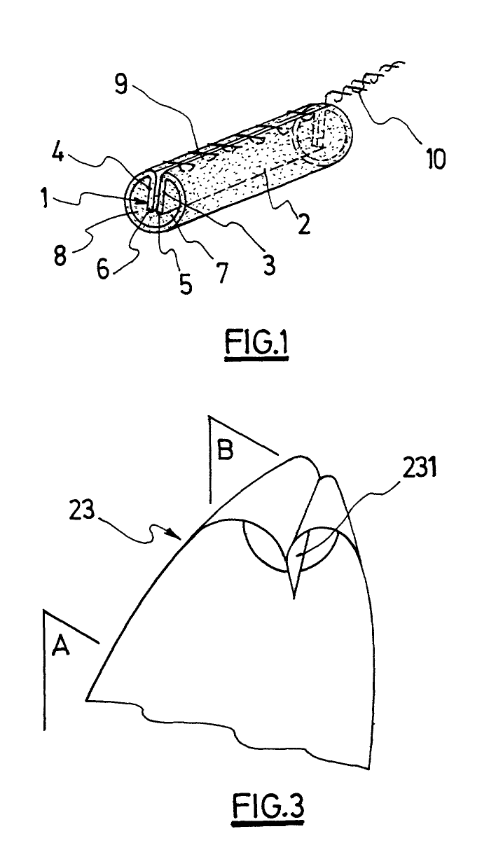

FIG. 1 is a perspective view of the dressing product;

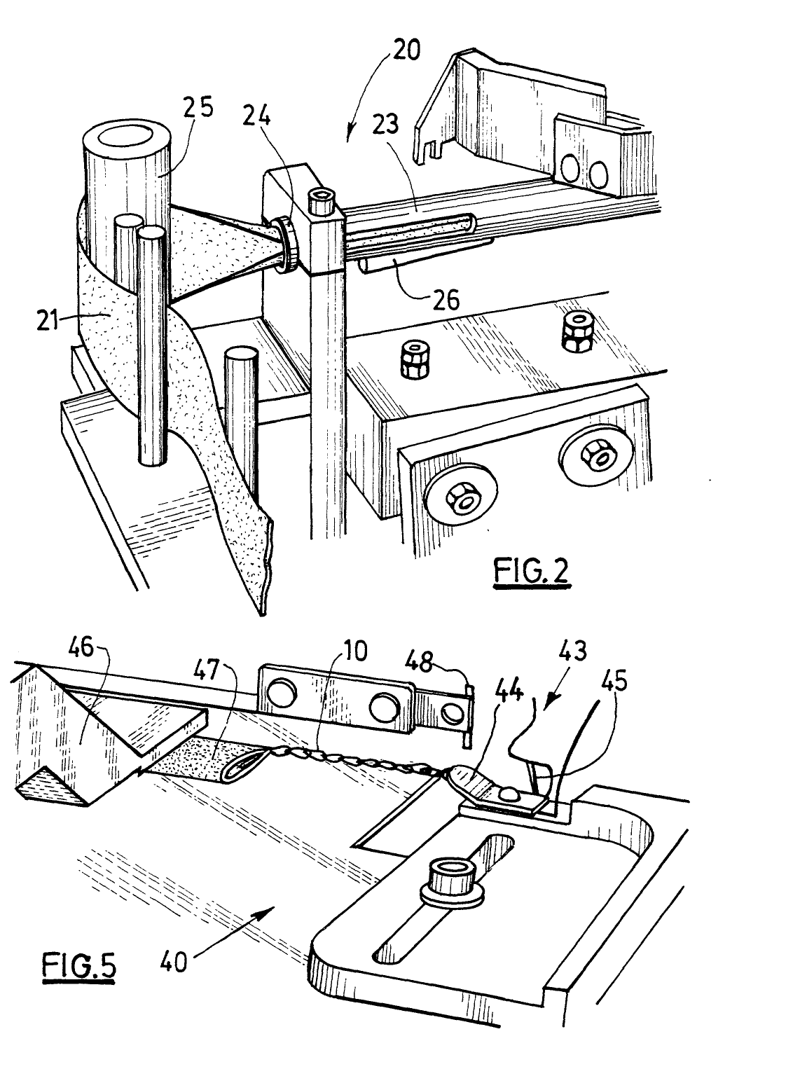

FIG. 2 is a perspective view from above of a part of the equipment shaping the fibre layer into a tube;

FIG. 3 is an internal perspective view of the shaping die;

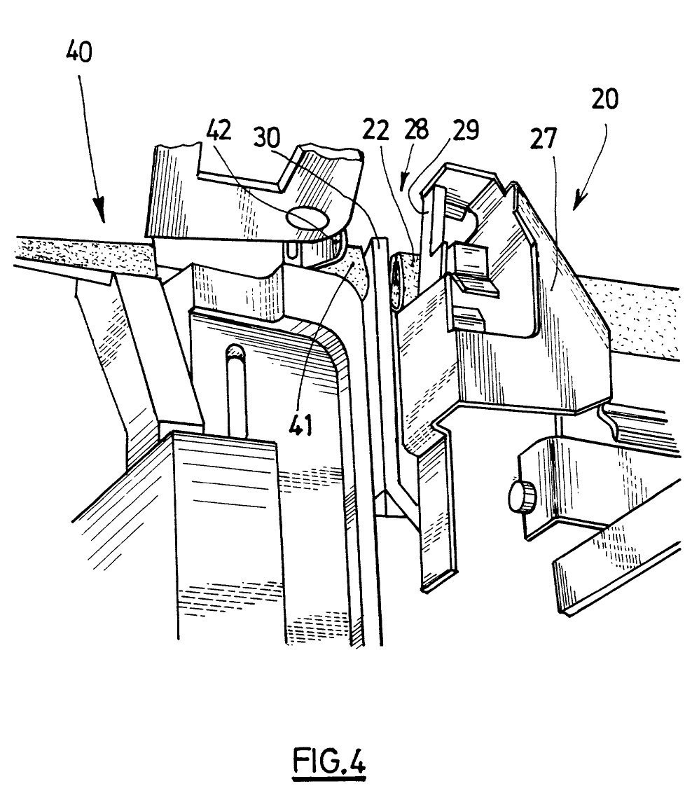

FIG. 4 is a perspective view from below of the shaping equipment and the overhand sewing machine, on the side opposite that in FIG. 2 and

FIG. 5 is a perspective view from above of the overhand sewing machine.

With reference to FIG. 1, the dressing product is composed of an alginate fibre layer, in this case, non-woven, rolled, folded back and oversewn.

In this way, the dressing comprises a core 1 and a sheath 2 extending from each other in a continuous layer. The core 1 is composed of the edges 3, 4, adjacent to the selvedges 5, 6, which were folded inwards after rolling the layer onto itself to form the sheath, which is tubular in this case. Two drainage slots 7, 8 extend longitudinally at either end of the core 1, between the core 1 and the sheath 2. The sheath 2 being roughly tubular in this case, the core 1 extends roughly in an axial plane, over two thirds of the transversal diameter of the sheath.

The sheath 2 is oversewn, i.e. the edges 3, 4 are joined together by overhand sewing 9, the overhand sewing 9 extending longitudinally beyond the sheath 2 into a pulling and safety cord or string 10.

To obtain a clearer idea, to produce a dressing with a sheath approximately 5 mm in diameter, the applicant started with a 22 mm width, for a sheath of 10 mm in diameter, of a 46 mm width. The dressing may be produced in an approximate length of 40 to 200 mm, with a cord approximately 40 to 100 mm in length.

The manufacturing method of the dressing will now be discussed. The manufacture is carried out using shaping equipment 20 and an overhand sewing machine 40.

The shaping consists, starting from a width 21, of forming a product which progressively takes the shape of an open tube, a tube with longitudinal edges which are drawn in and folded inwards followed by such a crushed tube 22, before being cut and pulled into the overhand sewing machine.

The shaping is performed in a guide or die 23, the width being already almost completely rolled onto itself at the entry 24 of the die. The rolled layer progresses in the die 23 under the effect of a rotary progression roller 25, outside the die and of an axis orthogonal to that of the die, and a compressed air stream introduced tangentially into the die, via a lateral nozzle 26, to “lubricate” the die and facilitate the progression of the layer being formed (FIG. 2).

With reference to FIG. 3, the axially progressive shape of the inner wall of the die 23 can be seen clearly, from a section A of open tube to a circular section B of tube closed onto itself with edges folded inwards, by means of the end median wall portion 231 in the form of a double upper side.

The tubular layer 22 coming out of the die 23 is treated and crushed by a clamp 27 before being taken into the overhand sewing machine 40 and cut by a cutting assembly 28 with a blade 29 and counter-blade 30 between the clamp 27 and the overhand sewing machine 40 (FIG. 4).

The tubular layer 41 preshaped before sewing in the overhand sewing machine is pulled into the machine by a traction base 42 (FIG. 4) to the overhand sewing station 43, with the sewing base 44 and the needle 45 (FIG. 5).

Under the effect of a pressing and translation base 46, the dressing product 47 produced in this way is pulled along the pulling and safety cord to be produced before the cord 10 is cut to the correct length with a cutting blade 48.

1. Dressing product for biological fluid wounds, comprising a metal alginate fibre core (1) surrounded by a supporting sheath (2) of a structure permeable to the biological fluid and biocompatible with the wound, characterised in that the core and sheath both extend into a continuous layer of alginate fibres and provide drainage slots (7, 8) between each other.

2. Product according to claim 1, wherein the alginate fibre layer is chosen in the group of non-woven, woven and knitted layers.

3. Product according to claim 1, wherein the sheath is composed of a tubular sheath (2) wherein the edges (3, 4) adjacent to the selvedges (5, 6) are folded inwards to form the core (1).

4. Product according to claim 1, wherein the core (1) extends at least over approximately a quarter of the transversal dimension of the sheath.

5. Product according to claim 3, wherein the edges (3, 4) of the sheath (2) are oversewn.

6. Product according to claim 5, wherein overhand sewing (9) is provided which extends beyond the sheath (2) and the core (1) into a pulling and safety cord (10).
